(19) United States
(12) Patent Application Publication
(10) Pub. No.: US 2025/0166744 A1
(43) Pub. Date: May 22, 2025

(54) SYSTEM AND METHOD FOR PREDICTIVE CANDIDATE COMPOUND DISCOVERY

(71) Applicants: Kenneth BEAN, Boynton Beach, FL (US); Paul RETLEWSKI, Cupertino, CA (US); Sujaiy SHIVAKUMAR, Hillsboro, OR (US)

(72) Inventors: Kenneth BEAN, Boynton Beach, FL (US); Paul RETLEWSKI, Cupertino, CA (US); Sujaiy SHIVAKUMAR, Hillsboro, OR (US)

(73) Assignee: FASTTRAC LLC, Laguna Beach, CA (US)

(21) Appl. No.: 18/832,598

(22) PCT Filed: Jan. 24, 2023
(86) PCT No.: PCT/US2023/011398

§ 371 Date: Jul. 24, 2024

Related U.S. Application Data
(60) Provisional application No. 63/302,418, filed on Jan. 24, 2022.

(51) Int. Cl. G16H 10/20; G06F 3/0482; G06T 15/00

(52) U.S. Cl. CPC G16H 10/20; G06T 15/00; G06F 3/0482; G06T 2200/24; G06T 2210/41

(57) ABSTRACT

A computing system for evaluating candidate molecules for use in candidate compound discovery is described. The system has a non-transitory computer-readable and a processor configured to receive a type of standardized data from at least a database, receive a user query, process each of the standardized data types with one or more trained machine learning models, generate an interactive environment comprising graphical representation of the candidate molecule and candidate compound based on the user query; based on a received a signal from a user, alter a configuration of the candidate compound to allow the user, using virtual reality (VR), augmented reality (AR) or both, to interact with the candidate compound and the plurality of candidate molecules and generate clinical characteristics of the candidate compound based on the further alteration by the user.

### CROSS-REFERENCE TO RELATED APPLICATION OF THE INVENTION

The present utility patent application is a United States National Stage application filed under 35 U.S.C. § 371 of International Patent Application No. PCT/US23/11398 filed on Jan. 24, 2023 System And Method For Predictive Candidate Compound Discovery, which claims the priority benefit of U.S. provisional patent application Ser. No. 63/302,418 filed on Jan. 24, 2022, entitled System And Method For Predictive Candidate Compound Discovery, the entirety of each is incorporated herein by reference for all purposes.

### TECHNICAL FIELD

The present invention generally relates to systems and methods for data retrieval, predictive analysis, and interactive visualization for discovering candidate compounds having certain attributes. More specifically, the present invention relates to an artificial intelligence assisted computer-implemented system and method for evaluating candidate molecules for use in candidate compound discovery, interacting with the compound in a virtual or augmented reality environment, and predicting clinical success.

### BACKGROUND

Compound development and discovery involves a massive amount of testing and analysis. Generally, a plurality of samples must be tested under a variety of conditions. The evaluation of a new compound must undergo numerous tests and trials before it is approved for use for its intended purpose, whether it be the discovery of new or unique drugs, nutraceutical compounds, chemicals, plastics, metals, alloys, and the like. In healthcare, the primary research is directed toward developing a new understanding of natural substances or physiological processes that produce desirable effects involves massive costs in research and development. In healthcare, research and development costs estimations for the development of new drugs range widely, from $43.4 million to $4.2 billion.

With technological advancements and understanding of biological systems, it is easier to predict new features of chemical, biological or metallic entities, with the help of various application software, hardware, and supercomputers. This is known as testing in silico. In silico attempts to develop new compounds with those currently made by identifying similar components and using behaviors known from previous compounds for new discovery. The component and its parts may be identified, recombined with other components, and tested in silico for desired effects.

Today, various approaches are employed in identifying biological, chemical, or physical attributes to compounds discovered and developed by applying large-scale computing technology. However, these approaches do not produce industry agnostic results efficiently or effectively, are onerous for the user, and are inaccurate as to certain features of a chemical molecule within a particular time, particularly because approaches employed today do not efficiently utilize the available information. Indeed, one of the most extensive challenges of testing candidate compounds and molecules is comparing information from disparate sources in different formats. In addition, using current systems, it is not possible to present consistent, accessible data across a sizeable extensive collection of data sources that comply with a typical industry data model having normalized or standardized parameters in data elements. Further, another drawback of current systems is that they do not capture the consistent relationships between sub-atomic particles and the candidate molecules and compounds to ensure accuracy of predictive models.

Further, many metals (e.g., sodium, potassium, magnesium, calcium, iron, zinc, copper, manganese, chromium, molybdenum and selenium) are required for normal biological functions in humans. Disorders of metal homeostasis and of metal bioavailability, or toxicity caused by metal excess, are responsible for a large number of human diseases. Metals are also extensively used in medicine as therapeutic and/or as diagnostic agents. Metals such as arsenic, gold and iron have been used to treat a variety of human diseases. Nowadays, an ever-increasing number of metal-based drugs are available. These drugs contain a broad spectrum of metals, many of which are not among those essential for humans, able to target proteins and/or DNA. As an example, metal-containing compounds targeting DNA or proteins currently in use, or designed to be used, as therapeutics against cancer, arthritis, parasitic and other diseases, with a special focus on the available information, are often provided by X-ray studies, about their mechanism of action at a molecular level. However, currently, when presented with these situations in the industry, it is difficult and unduly expensive to identify and test metals for therapeutics.

In light of the above-mentioned problems, there is a need for a system and method that allows a user to evaluate candidate molecules for use in candidate compound discovery, interact with the compound in a virtual or augmented reality environment, and predict clinical success in a user-friendly way.

### SUMMARY OF THE INVENTION

The present invention generally discloses a system and method for gathering, assembling, sorting, and retrieving data. Further, the present invention relates to a computing system for evaluating candidate molecules for use in candidate compound discovery, applying a multitude of artificial intelligence (AI) models, generating interactive virtual environments in atomic resolutions to allow a user to test candidate molecules with candidate compounds. The AI implemented system is configured to gather and receive information relating to, for example, physical, chemical, electrical, genetic, atomic, sub-atomic and biological dimensions of molecules, metals, and compounds using disparate types of information such as all literature in both structured and unstructured formats and reconfigured to identify the similarities and dissimilarities in their composition as well as in their atomic and sub-atomic behavior.

In embodiments, the system and method use a plurality of artificial intelligence (AI) models and virtual reality (VR) or augmented reality (AR) for compound discovery. AR offers several advantages over traditional visualization tools (2D computer projections, 3D computer projections, and 3D printed plastic models), including but not limited to, allowing researchers to interact with the molecules in ways that simulate their natural environment. For example, using the system and method, a researcher (or user) may simulate an enzyme in the presence of its ligand and view the structure, allowing the researcher/user to examine the steric changes that occur within the molecule upon ligand binding. Additionally, the system and method may use VR/AR to understand how a known drug may interact with the molecule and output potential clinical attributes of the candidate compound. Furthermore, researchers (or users) may effectively view and interact with molecules on an atomic and sub-atomic scale. In sum, VR/AR coupled with the system described herein allows researchers to rationally design new drug candidates and test these in silica for binding and fit before investing in drug development and manufacturing. In effect, the system can automatically test many thousands of molecules in silica, then allow a user to interact with plurality of suggested molecules and candidate compounds using VR/AR space to understand critical interactions, eventually leading into in vivo testing.

In embodiments, a discovery system or computer-implemented system that may be integrated with or in communication with a plurality of embedded AI models (e.g., neural networks, RNN, CNN, NLP) in a networked or virtual networked environment is presented. In one embodiment, the system is configured to retrieve standardized data of compounds and molecular information of compounds down to the atomic and sub-atomic level, compare the known physical, chemical, electrical, genetic, atomic, sub-atomic and biological characteristics of molecules and identify potential new combinations of molecules based on similarities in their composition and chemical and atomic characteristics, based in part on known actions of similar compounds and elements. In one embodiment, the software application outputs results based on a plurality of AI-aided user filters. The comparison is made using the system based on accelerating time for researchers performing research activities. In one embodiment, the system is installed with application software, mobile application, or web-based application.

In one embodiment, the system after analysis is configured to use a combination of AI models to predict at least one or a plurality of optimized molecules which could be added to a compound to achieve a desired clinical attribute and view the comparative analysis in drug discovery in a three-dimensional interactive environment using VR or AR. The user can visualize the compound's molecular structure and make changes based on hand gestures or other types of user inputs (e.g., brain waves). In one embodiment, the system comprises a computing device having a processor and a memory in communication with the processor. The memory stores a set of instructions or software modules executable by the processor. In one embodiment, the software modules may be application software, mobile application, or web-based application residing on a server or as part of a virtual network. In one embodiment, the system further comprises a database management system. In one embodiment, the database management system comprises one or more databases in communication with the computing device configured to store a plurality of standardized data of various compositions, compounds, metals, molecules, atoms and sub-atomic particles. In one embodiment, the data management system includes one or more modules configured to analyze, predict, and recommend specific molecules or elements for various desired compounds to give the effect required by the user (e.g., certain attributes or clinical attributes). The efficacy prediction may be based on similar compositions sharing typical biological receptors, enzymatic pathways, chemical structures, and the like.

In one embodiment, the system generates a visual representation configured to compare the known physical, chemical, genetic, stereoisomerism, empirical formula, Valence Shell Electron Pair Repulsion (VSEPR), and biological dimensions of molecules, and in a second layer, a visual representation of characteristics of metals that may be added. It is configured to identify the similarities and behavior in the respective molecular compositions, thereby providing a comparative visualization of analyzed results, having identified similar composition and similarities in their behavior concerning the existing composition.

The present system's visualization layer (interface) will present data in a graphical format where hundreds of parameters can be viewed simultaneously. A detailed drill-down inspection can be performed at any parameter for candidates being investigated through the use of VR and/or AR overlays, which allow researchers to quickly view actual data (not just a graphical representation) and save the findings of interest in a working file for later review. The system's ability to quickly investigate all the parameters required to review in a highly accelerated fashion is novel to the industry and as a result of this efficiency, use of the program will accelerate traditional research time by an anticipated magnitude factor of 2 (up to 100× faster than traditional efforts).

In one embodiment, the comparison is performed on any level of scale, in vivo, in vitro, and in silico simultaneously. The results of comparison self-checks for accuracy, using available data repositories. The comparison is made using the system based on accelerating time for researchers performing research activities. In one embodiment, the system is installed with application software, mobile application, and/or web-based application.

In one embodiment, the software with artificial intelligence will provide those same characteristics to the pharmaceutical explorer, suggesting paths of chemical and/or structural modifications, while also suggesting targets, which will significantly improve the efficiency of drug discovery and development. In one embodiment, the system utilizes the fast-processing capabilities of graphical processing units (GPUs) and supercomputers together with algorithms for similarity detection. In one embodiment, the algorithms are trained using machine learning to identify high potential similarity matches accurately as well as dissimilarities, and the results are displayed to the users using 3D, augmented reality visualizations.

In one embodiment, the software and artificial intelligence are used to predict features of new chemical or biological entities. The artificial intelligence coupled with predictive analytics evolves from predicting solubility, toxicity, and antigenicity, to services incorporating prediction of efficacy based on similar compounds and entities sharing common biological receptors, enzymatic pathways, or chemical structures. It provides value to the very crucial “go/no go” decision making and makes actual engineering modification of the molecules, which could improve both efficacy and limit or reduce toxicity, including antigenicity in the case of protein-based drugs such as antibodies.

In one embodiment, one or more modules include a data platform system (DPS), an artificial intelligence system (AIS), and a molecule and compound editor system (MCEM). In one embodiment, the DPS is integrated with a user interface (UI) configured to store at least one of the standardized and normalized data related to compounds (drugs and chemicals), which provides administration and functions for real-time monitoring of users and their data environments. In one embodiment, the DPS supports data in the form of texts, graphical data, functions, equations, formulas, 2D or 3D figures, and models corresponding to atomic, chemical, physics, biological, mathematical representations, and abstracts for storing and accessing data.

In one embodiment, the artificial intelligence module (AIM) is configured to provide a multitude of AI modules having a plurality of AI methodologies and techniques to compare the candidate compound with at least one of the standardized and normalized data related to similar compounds through at least one of the AI modules of the AIS. The AIM outputs the results of the AI modules based on a plurality of inputs. In one embodiment, each AI module includes natural language processing (NLP), Neuro Nets (NL), Machine learning (ML), recurrent neural network (RNN), and Convolutional Neural Network (CNN).

In one embodiment, the molecule and compound editor system (MCEM) are configured to analyze the predicted results upon user direction by utilizing analysis software of known compounds and/or metals. In one embodiment, the AIM is configured to return potential hit molecules that may be candidate molecules and/or metals for a requested candidate compound upon request function for analysis in the MCEM. The analyzed results from the AIS, MCEM are stored in the DPS, and its process logistics are recorded in the FTDS for further processing. In one embodiment, the comparative visualization displays result in a graphical format to users using three-dimensional and or augmented reality (AR) visualizations simultaneously.

In one embodiment, the software with artificial intelligence (AI) provides those same characteristics to the compound (e.g., pharmaceutical or chemical) or metal explorer, suggesting paths of chemical and structural modifications, suggesting targets, which will significantly improve the efficiency of compound manufacturing and/or drug discovery and development. In one embodiment, the system utilizes the fast-processing capabilities such as dedicated AI configured GPUs, supercomputers, or quantum computing together with algorithms for similarity detection. In one embodiment, the algorithms are trained using machine learning to accurately identify high potential similarity matches, and the results are displayed to users using virtual and augmented reality visualizations.

In one embodiment, the software and artificial intelligence are used to predict features of new chemical or biological entities in some cases having metals inculcated therein. Artificial intelligence coupled with predictive analytics has evolved from predicting solubility, toxicity, and antigenicity, to services incorporating prediction of efficacy based on similar compounds and entities sharing common biological, organic receptors, enzymatic pathways, or chemical structures and metallic properties. It provides value to the very crucial “go/no go” decision making as well as to the actual engineering modification of the molecules, which could improve both efficacy and limit or reduce toxicity, including antigenicity in the case of protein-based drugs such as antibodies or in the case of chemicals, efficacy and safety.

An exemplary output of the software is a modified receiver operator curve; the ordinate shall be a value generated from a regression combining available evidence for similar metals and would include a rating of how similar the innovator metal was concerning the existing metal(s). The abscissa would indicate the predictor of success, from 0 to 100. An additional characteristic would consider the number of tests and data available for each comparison. Metals with only chemical resistance data would have a higher risk associated as a caution with the predictor compared, for example, with a chemical resistance, conductivity, density, and past uses cases.

A computing system for evaluating candidate molecules for use in candidate compound discovery, the computer system comprising a non-transitory computer-readable memory, and a processor configured to execute instructions stored on the non-transitory computer-readable memory which, when executed, causes the processor to receive a type of standardized data from at least a database, wherein the standardized data is for compounds, metals, molecules, atoms, sub-atomic particles or any combination thereof, and wherein the standardized data types comprise numerical data sets, images, graphs, text, or any combination thereof, receive a user query, wherein the user query comprises a request for a desired attribute for the candidate compound, processes each of the standardized data types with one or more trained machine learning models, wherein the one or more machine learning models are configured to recommend at least one of a plurality of the candidate molecules to be bonded with the candidate compound based on the user query, generates an interactive environment comprising a graphical representation of the candidate molecule and candidate compound based on the user query, then based on a received signal from a user, alters a configuration of the candidate compound to allow the user, using virtual reality (VR), augmented reality (AR), or both, to interact with the candidate compound and the plurality of candidate molecules, wherein based on a received additional signal from the user, the computer system can further alter the candidate compound with another of the plurality of molecules, and generate clinical characteristics of the candidate compound based on the further alteration by the user.

In embodiments, a computer-implemented method candidate compound discovery, comprising executing on a processor the steps of receiving a type of standardized data from at least a database, wherein the standardized data is for compounds, metals, molecules, atoms, sub-atomic particles or any combination thereof, and wherein the standardized data types comprise numerical data sets, images, graphs, text, or any combination thereof, receiving a user query, wherein the user query comprises a request for a desired attribute for the candidate compound, processing each of the standardized data types with one or more trained machine learning models, wherein the one or more machine learning models are configured to recommend at least one of a plurality of the candidate molecules to be bonded with the candidate compound based on the user query, generating an interactive environment comprising graphical representation of the candidate molecule and candidate compound based on the user query, based on a received a signal from a user, altering a configuration of the candidate compound to allow the user, using virtual reality (VR), augmented reality (AR), or both, to interact with the candidate compound and the plurality of candidate molecules, wherein based on a received additional signal from the user, the computer system can further alter the candidate compound with another of the plurality of molecules, and generate clinical characteristics of the candidate compound based on the further alteration by the user.

In embodiments, a non-transitory computer-readable medium having stored thereon program instructions that, upon execution by a computing device, causes the computing device to perform operations comprising of receiving a type of standardized data from at least a database, wherein the standardized data is for compounds, metals, molecules, atoms, sub-atomic particles or any combination thereof, and wherein the standardized data types, comprising numerical data sets, images, graphs, text, or any combination thereof, receive a user query, wherein the user query comprises a request for a desired attribute for the candidate compound, processes each of the standardized data types with one or more trained machine learning models, wherein the one or more machine learning models are configured to recommend at least one of a plurality of the candidate molecules to be bonded with the candidate compound based on the user query, to generate an interactive environment comprising a graphical representation of the candidate molecule and candidate compound based on the user query, and based on a received signal from a user, alter a configuration of the candidate compound to allow the user, using virtual reality (VR), augmented reality (AR), or both, to interact with the candidate compound and the plurality of candidate molecules, wherein based on a received additional signal from the user, the computer system may further alter the candidate compound with another of the plurality of molecules and generate clinical characteristics of the candidate compound based on the further alteration by the user.

Advantageously, the systems and methods transform different types of standardized data into data usable to make predictions based on various comparisons. Standardized data comes in the form of numerical data, images, graphs, and literature, and by converting these data sets using various AI models, the efficacy of predictive molecules for use is significantly increased.

Moreover, the time and financial constraints in compound development is significantly decreased. The system is configured to utilize empirical data on features such as how certain molecules may impact the body, such as one's body temperature, for example. The system is configured to predict effective metals to use in therapeutics in a second layer by parsing characteristics such as but not limited to, crystalline structure, and chemical and physical properties, both from predictive software, empirical data, and published data to develop a multidimensional modeling system to improve the accuracy of the model and, in turn, increase successes designing new metals and suggesting new applications for those metals.

Advantageously, the system removes any subjective input in favor of more objective appraisals when applied to the selection process and “go/no go” decisions in candidate compound discovery.

Advantageously, the platform provides the ability for quick, practical working sessions. The system will have the ability to provide cognitive comparison tools by optimizing interfaces so that a researcher can quickly analyze the data. The system will have an immersive set of tools that work in conjunction with each other for comparison, applying and alternating filters for viewing data, saving/recalling work sessions notes and findings, and executing detailed AI analysis across known and new molecules that are being developed.

The interface may be intuitive and utilizes gestures (either through using a mouse, hand controllers, key presses, or other means), which will become second nature to the researcher. The ability to pivot quickly is paramount to the design effort for the system. The need to identify source data, collect information, and prepare it before analyzing it is eliminated. The data will be available for the researcher to use, and he or she will be able to concentrate on the research at hand.

Advantageously, the system is configured to ingest and transform data from disparate sources and takes a continual effort to collect, assemble and parameterize the data in a consistent and consumable manner. The resultant data repository is traceable to source through logging of source and transformation techniques for reliability by researchers across a large body of information.

Advantageously, using scalable architectures in both elastic cloud data centers, use of highly performing graphical processing units (GPUs), the system distributes computing workload for demand across multiple computing processors. Due to the enormity of data, the system provides data processing at various levels such as real-time highly performant (elastic), real-time fixed capacity, and off-peak lower-cost processing, such as overnight or with lower-performing/low-cost CPU allocation.

Advantageously, the system is able to provide clear views of compounds, molecules and sub-atomic particles in an interactive environment at a biological level of 10⁻⁶.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 is a block diagram of a computer-implemented system integrated with artificial intelligence in one embodiment of the present invention.

FIG. 2 is a block diagram illustrating data mining by an AI module in one embodiment of the present invention.

FIG. 3 is a block diagram of a system with one or more features allowing a user to communicate with the cloud platform in one embodiment of the present invention.

FIG. 4 is a block diagram of a system illustrating client and server-side system architecture for evaluating candidate molecules according to one embodiment of the present invention.

FIG. 5 is a block diagram of a system illustrating the communication between the user interface and server slot in one embodiment of the present invention.

FIG. 6 is a block diagram illustrating a data platform system (DPS) for storing drug information in one embodiment of the present invention.

FIG. 7 is a block diagram illustrating an artificial intelligence system (AIS) for processing analyzed data of the FTDS system in one embodiment of the present invention.

FIG. 8 is a block diagram illustrating an exemplary data mining operation in one embodiment of the present invention.

FIG. 9 is a block diagram of an exemplary system architecture and data routing scheme in an embodiment of the present invention.

FIG. 10 is a blocking diagram of an exemplary virtual network for use in embodiments of the present invention.

FIG. 11 is a Venn-style diagram showing similarity overlap according to embodiments of the present invention.

FIG. 12 is a block diagram illustrating the modes of data gathering, transformation and building an interactive environment in one embodiment of the present invention.

FIG. 13 is a block diagram of an exemplary system architecture for building a virtual interactive environment in embodiments of the present invention.

FIG. 14 is a block diagram of an exemplary user management architecture in embodiments of the present invention.

FIG. 15 is an exemplary illustration of a user interacting with a user interface on a computer and a user interacting with the interactive environment in embodiments of the present invention.

FIG. 16 is another exemplary illustration of a user interacting with the interactive environment in embodiments of the present invention.

FIG. 17 is an exemplary user interface showing an interactive molecule and user interface with selectable parameters in embodiments of the present invention.

FIG. 18 is an illustration of a user interacting in the environment with a molecule and compound in embodiments of the present invention.

FIG. 19 is a flow chart showing a recommendation system in embodiments of the present invention.

FIG. 20 is an exemplary user interface showing an interactive molecule and user interface with a data set provided by the system in embodiments of the present invention.

FIG. 21 is a molecular diagram showing a compound and then, a candidate compound together with a bonding map in embodiments of the present invention.

FIG. 22 is an electron orbit map showing subatomic visualization in a user interface in embodiments of the present invention.

FIG. 23 is an exemplary compound visualization showing potential moldy complexes in embodiments of the present invention.

FIG. 24 is an exemplary molecular diagram showing how an insertion of a candidate molecule may cause a condition that is realized by the system in embodiments of the present invention.

FIG. 25 is a molecular diagram showing symmetrical and asymmetrical chemical bonding in embodiments of the present invention.

### DETAILED DESCRIPTION OF EMBODIMENTS

The present invention is best understood by reference to the detailed figures and description set forth herein.

It is understood that the present subject matter may be embodied in many different forms and should not be construed as being limited to the embodiments set forth herein. Rather, these embodiments are provided so that this subject matter will be thorough and complete and will convey the disclosure to those skilled in the art. Indeed, the subject matter is intended to cover alternatives, modifications, and equivalents of these embodiments, which are included within the scope and spirit of the subject matter as defined by the appended claims and their equivalents. Furthermore, in the detailed description of the present subject matter, numerous specific details are set forth in order to provide a thorough understanding of the present subject matter. However, it will be clear to those of ordinary skill in the art that the present subject matter may be practiced without such specific details.

Aspects of the present disclosure are described herein with reference to flowchart illustrations and/or block diagrams of methods, apparatuses (systems), and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

The non-transitory computer-readable media includes all types of computer-readable media, including magnetic storage media, optical storage media, and solid-state storage media and specifically excludes signals. It should be understood that the software can be installed in and sold with the device. Alternatively, the software can be obtained and loaded into the device, including obtaining the software via a disc medium or from any manner of network or distribution system, including, for example, from a server owned by the software creator or from a server not owned but used by the software creator. The software can be stored on a server for distribution over the Internet, for example.

Computer-readable storage media (medium) exclude (excludes) propagated signals per se, can be accessed by a computer and/or processor(s), and include volatile and non-volatile internal and/or external media that is removable and/or non-removable. For the computer, the various types of storage media accommodate the storage of data in any suitable digital format. It should be appreciated by those skilled in the art that other types of computer readable medium can be employed such as zip drives, solid state drives, magnetic tape, flash memory cards, flash drives, cartridges, and the like, for storing computer executable instructions for performing the novel methods (acts) of the disclosed architecture.

The computing system includes data stores, which maintain a database according to known database management systems (DBMS). The data stores may include a hard disk drive, a magnetic disk drive, an optical disk drive, or another type of computer readable media which can store data accessible by the processor, such as magnetic cassettes, flash memory cards, digital versatile disks, cartridges, random access memory (RAM) and read only memory (ROM). The data stores may be connected to the computing system bus by a drive interface and the data stores provide nonvolatile storage of computer readable instructions, data structures, program modules and other data for the computing system.

The terminology used herein is for the purpose of describing particular aspects only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

For purposes of this document, each process associated with the disclosed technology may be performed continuously and by one or more computing devices. Each step in a process may be performed by the same or different computing devices as those used in other steps, and each step need not necessarily be performed by a single computing device.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, “modules” refers to computer program logic implemented in hardware, firmware, or software. In some examples, modules can be stored on a storage device, loaded into the memory and executed by processor, or be part of a virtual network. For example, modules ca be implemented as one or more computer programs, i.e., one or more modules of computer program instructions, encoded on a tangible computer storage medium for execution by one or more processors.

As used herein, “standardized data” refers to publicly trusted and available data relating to molecules compounds atomic elements, subatomic particles in the like. The data may include and is not limited to information relating to bond angles, electron configurations, melting points, toxicity, efficacy, physical, chemical, genetic, anatomical, biological dimensions and the like.

As used herein, “images” refers to non-numerical data such as molecular images.

As used herein, “desired clinical attribute” or “desired attribute” refers to a request by a user to the system for a characteristic of a molecule or compound (e.g., affinity) or a molecule or compound that may result in treating a disease or condition.

As used herein, “candidate compounds” refer to a compound that is being built by the user to treat a disease or condition.

As used herein, “candidate molecule” refers to molecules that may bond to the compound to provide a desired clinical attribute and may also refer to atomic elements that could be added to a candidate compound.

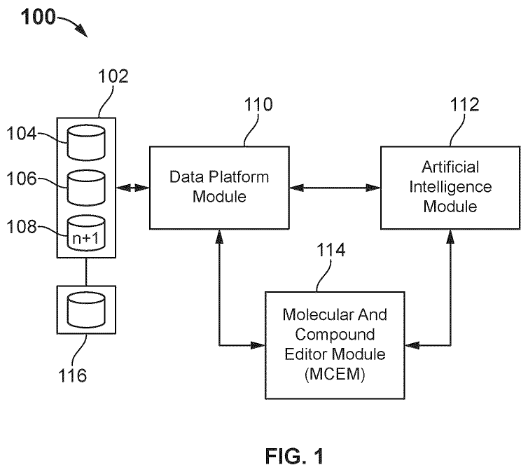

Referring to FIG. 1, a block diagram illustrating a system for predicting new compounds discovery computer-implemented system 100 integrated with artificial intelligence (AI) presented in a networked environment, according to one embodiment of the present invention. In one embodiment, system 100 comprises a computing device having a processor and a memory in communication with the processor, wherein the memory stores a set of instructions or software modules executable by the processor. In one embodiment, the software module could be any application software, mobile application, and/or web-based application.

In one embodiment, system 100 further comprises a database management module 102 in communication with one or more databases (104, 106, 108 and 116). The databases (104, 106, 108 and 116), are configured to store standardized data and information of various compounds, metals, molecules, atoms, and subatomic particles as an example (e.g., all known compositions). The database management module 102 is configured, in exemplary embodiments, to scrape predetermined information hubs that are peer reviewed and trusted sources for the information, including but not limited to, chemical characterizations, physical attributes, mathematical calculations, formulas, and values. In embodiments, the system is configured to leverage tabulated data, written/text literature, graphical representations, equations, figures, and parameters. In embodiments, the information includes, but is not limited to, details of physical, chemical, genetic, atomical, and biological dimensions of molecules isolated from each other in an organizable manner incorporating, biological receptors, enzymatic pathways, or chemical structures. This data is received on the databases for use and transformation by the artificial intelligence module (AIM) 112.

In one embodiment, the AIM 112 is in communication with the data platform module 110, and is configured to provide plurality of AI models, methodologies and techniques to transform the standardized data into data sets usable by the system for purpose of molecule recommendation. Further, AIM 112 is configured to recommend at least one of a plurality of candidate molecules to the user based on a user query and desired attribute. In this way the AIM 112 is working in parallel to not only transform in parts data, but also to make predictions and recommendations to a user.

Referring still to FIG. 1, the system 100 further comprises a molecule and compound editor module (MCEM) 114 in communication with the DPM 110 and AIM 112. MCEM 114 is configured to generate an interactive environment comprising graphical representations of candidate molecules in atomic resolution based on certain user queries. Further, based on inputs from the AIM 112, the MCEM 114 is configured to alter a configuration of the candidate compound to allow a user using virtual or augmented reality to interact with the candidate compounds and candidate molecules in the plurality of candidate molecules based on received signals from the user via handheld VR and/or AR components, for example. The MCEM 114 utilizes inputs from the user, the data platform module 110, and the AIS 112, to generate a proper interactive environment while also utilizing the suggested molecules from the AIS 112. MCES 114 is configured to generate molecules in atomic resolution including the protons, neutrons, and electrons of atomic elements included therein.

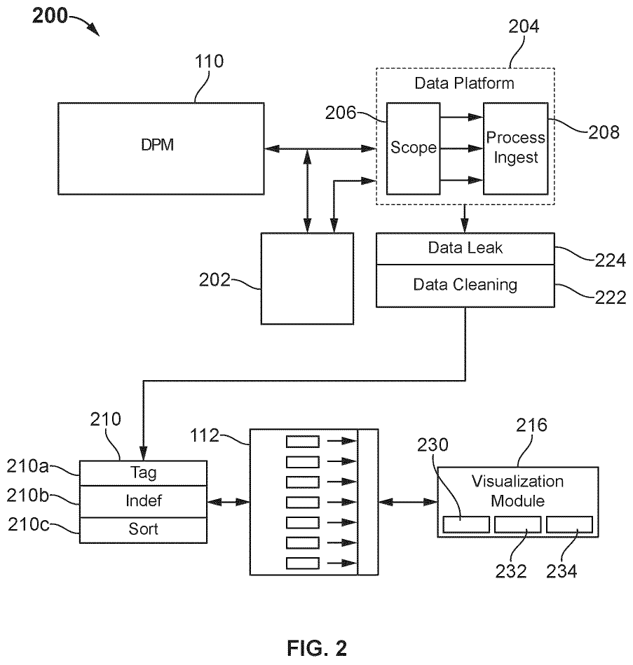

Referring now to FIG. 2, a block diagram 200 illustrating data gathering and mining via AIM 112, according to one embodiment of the present invention, is shown. In one embodiment, the DPS 110 is in communication with data platform 204, which comprises scraper 206 configured to extract data from outputs generated from other programs, and a process ingest 208 configured to move data from one source to another for analysis. In one embodiment, central hub 202 provides a collection of tools that facilitate data gathering. In one embodiment, the ingest 208 is in communication with a data cleaning 222 and a data leak 224. Prior to evaluation by the AIS 112, data leak 224 and data cleaning 222 are configured to separate and remove non-functional data or data that is corrupted.

The data is then routed to a data parser 210, which is configured to tag 210a and index 210b and sort the standardized data 210c sets in preparation for processing by the AIS 112. The processed data results are displayed in visualization module 216, which includes the interactive environment 230, graphical representations 232 and drop-down menus 234 for the users to interact with.

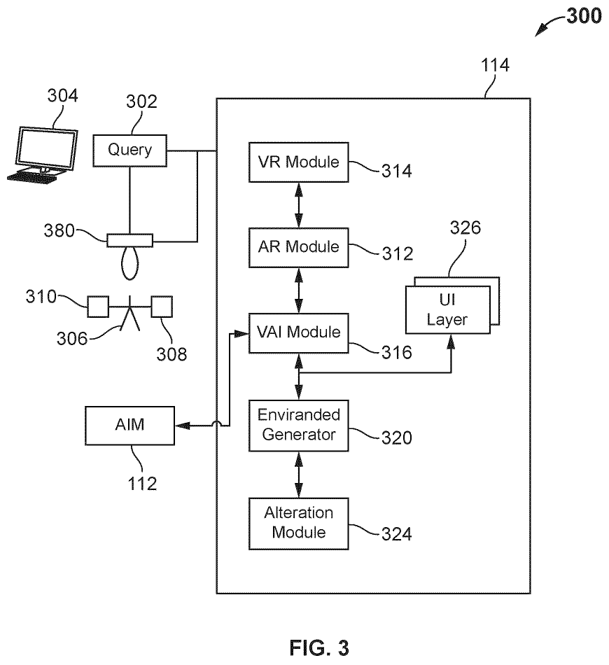

Referring now to FIG. 3, a block diagram of the MCEM 114 is shown generally at 300. The MCEM 114 comprises AVR module 314, AR module 312, VAI module 316, environment generator 320, alteration module 324, and UI layer 326. The MCEM 114 is in communication with the AIS 112. In operation, the MCEM 114 is configured to generate an interactive environment comprising graphical representation of candidate molecules in atomic resolution based on user query 302. The user query 302 may be an output from a client device 304 or a user 306 having virtual or augmented reality handheld devices 308 and 310.

VR module 314 is configured to execute based on the user query and AR module 312 is configured to execute based on the user query. The environment generator 320 is configured to generate graphical representations of compounds and molecules in atomic resolution while allowing alteration module 324 to receive signals from the user to move the molecule or compound around the UI. In this way the user 306 can interact with the molecule and the compounds to view bond angles or sub-atomic particles to ascertain which molecule may be employed based on the desired clinical attribute, which are sorted at UI layer 326 for viewing in either the client display device 304 or the AR/VR apparatus 350.

The AIM 112 and the VAI module 316 are configured to receive inputs from the MCEM 114 and generate an AI assisted first user interface where, in operation, subsequent the user input or query, loads a menu on the user interface with recommended molecules based on the user input and order the recommended molecules in menu based on a predictive success parameters using trained data sets. The AR/VR apparatus 350 worn by the user allows the user to view the interactive environment with AR/VR headset.

In operation, the MCEM 114 is configured to display the result of the analyzed data for candidate molecule and compound bio-functionality. In one embodiment, the displayed result comprises details of identified similarities and is configured to deliver the corresponding values.

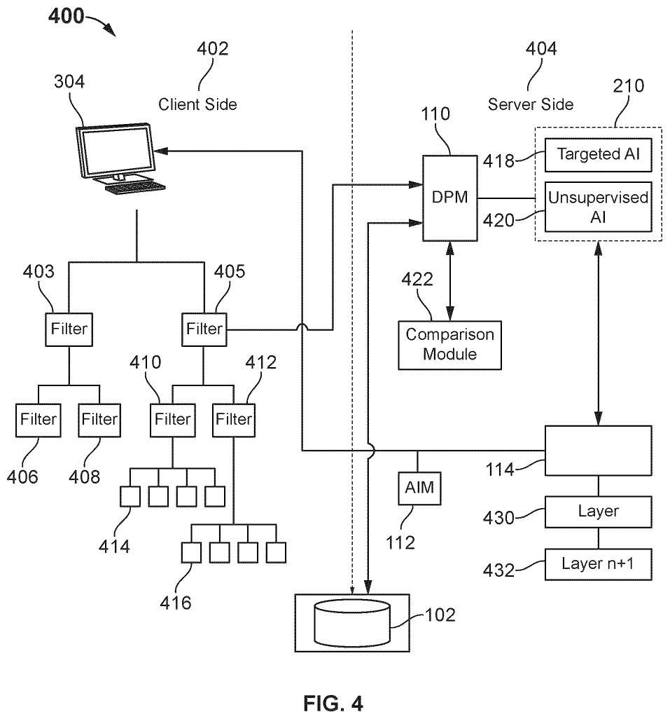

Referring to FIG. 4, a block diagram of a system 400 illustrating client and server-side system architecture for evaluating candidate molecules according to one embodiment of the present invention is shown generally at 400 in which users are assisted by AI in making filter selections to find a molecule that is best suited for the chosen clinical attribute. System 400 comprises a client-side 402 and a server-side 404. The server-side 404 is in communication with the client-side 402 via a network or cloud and the server-side 404 comprises a DPM 110 in communication with an AIM 112 and a comparison module 422. The client-side 402 comprises a plurality of filters (402-416) to request specific features of a candidate compound and further inspect the data at any parameter using an augmented virtual reality after the analysis and predictions are made. In one embodiment, virtual or augmented reality allows researchers to quickly view actual data (not just a graphical representation) and save the findings of interest in a working file for later review. The AIM 112 and comparison module 422 are configured to operate together to generate an AI assisted first user interface based on the user filters 401, 403, 405, 406, 408, 412, 414 and 416, a menu on the user interface with recommended molecules based on the user input and further, allows the user to choose a molecule in the ordered recommended molecules list in the menu based on a predictive success parameter using trained data sets. Filters may comprise user query choices such as biological, physical or chemical parameters. With each user chosen filter, the AIM 112 and comparison module 422 are configured to provide predictive elements based on success parameters. Further, the layers 430 and 432 may be provided in communication with the visualization module 216 and are configured to allow the user to input certain metals into the candidate compounds based on a plurality of variables taken together using targeted AI 418 or unsupervised AI 420 to generate additional predictive molecules to be chosen by the user based on the desired clinical attribute.

The comparison module 422 is configured for comparing the user request data, via filters, with the existing data present in database 102. In one embodiment, the comparison module 422 utilizes a plurality of AI models to perform the metric analysis techniques and optimize classifications. Then, in operation, the visualization module 114 displays the identified compounds or molecules in an interactive environment the client-side display 304.

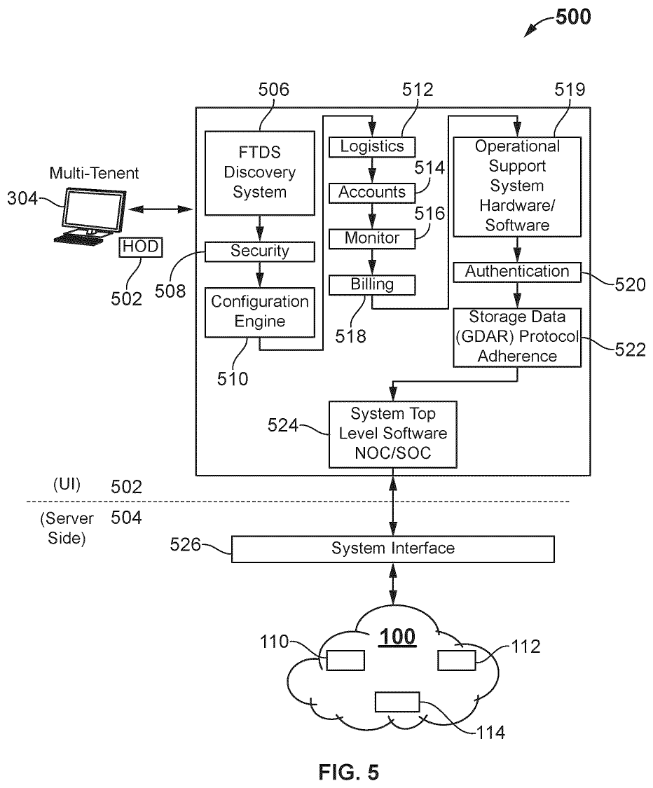

Referring now to FIG. 5, a block diagram of a system 500 illustrating the communication between the user interface (UI) and a server slot in one embodiment of the present invention is disclosed. The users can utilize a multi-tenant system that separates client data from shared data (repository and algorithms) to maintain the privacy concerns for clients and users and allow them to work together as needed. System 500 comprises a client display 220 configured to perform various operations, such as monitoring and billing. In one embodiment, system 500 comprises the discovery system (FTDS) 506, a security module 508, a configuration engine 510, a logistic module 512, an accounting module 514, a monitoring module 516, a billing module 518, an authentication layer 520, a storage data (GDAR) protocol adherence 522, and a system top-level software NOC/SOC 524. In one embodiment, the system 500 further comprises a server 502 having protocol APIs 526 in communication with the FTDS system 100 of the present invention. The system top-level software NOC/SOC 524 communicates with the system interface (protocol, API's) 526 via a user interface (UI) 502.

In one embodiment, system 100 provides all top-level administration, logistics, and direction for the entire software system. In one embodiment, the system 100 provides the custom user interface (UI) based upon user's criteria through a selection table that can be customized depending on the top-level selection, for example, compound/drug discovery, compound evaluation, etc. In one embodiment, system 100 provides the required security procedures and steps to conform with any and all security requirements. In one embodiment, the security is established between users and from external. In one embodiment, system 100 is the only portal from which users can use. In one embodiment, all the user inputs are processed by the system 100 directs data to other modules while monitoring the other systems. In one embodiment, system 100 also includes the ability to verify the user-selected function was processed and completed independently using Telco software. Further, system 100 includes AI to provide suggestions at all steps that are not part of the AIS.

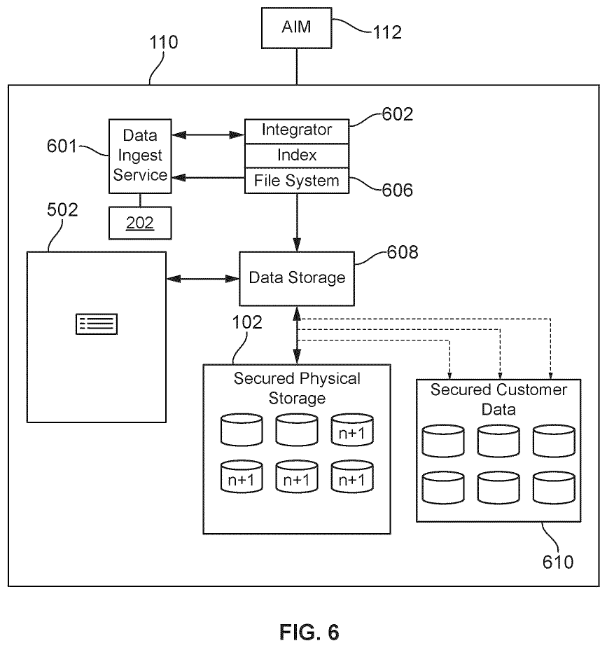

Referring to FIG. 6, a block diagram illustrates a data platform (DPS) for storing compound information in one embodiment of the present invention is disclosed. The DPS 110 comprises a data ingest service (DIS) 601 which comprises a collection of tools for gathering and transforming data. The DIS 601 is in communication with scraper 202, an integrator or integrator service 602, an index or index service 604, and a file system service 606. The DIS 601 is configured to prepare the data for use at the AIS 112 (e.g., transform the data gathered into usable sets and parameters through techniques such as tokens, labels, and metadata that optimize data retrieval). In one embodiment, the DPS integrator 602, index 604, and file system 606 use a control signal for retrieving data from the DIS 601. In one embodiment, the integrator 602 assembles the data as requested by the MCEM 110 and AIM 112, including the molecule/compound name and associated parameters, etc.

In operation, an integrator or integrator service 602, an index or index services 604 and a file system service 606 are configured to tag, index, and assign value to each of the data sets based on the user query. These modules are in communication with the AIS 112 and are configured to transform standardized data sets based on the chosen artificial neural network for rapid retrieval. The AIS 112 can be used to assist the index or index services 604 in tagging and indexing each of the data sets for retrieval.

Further, the DPM 110 allows multiple customer databases 610 with individual security protocols to isolate each database to support the service level agreement (SLA) for each customer. The DPM 110 service provides a real-time monitoring system showing the users and their data environments with safeguards and alarms to signal any issues with the system and/or users.

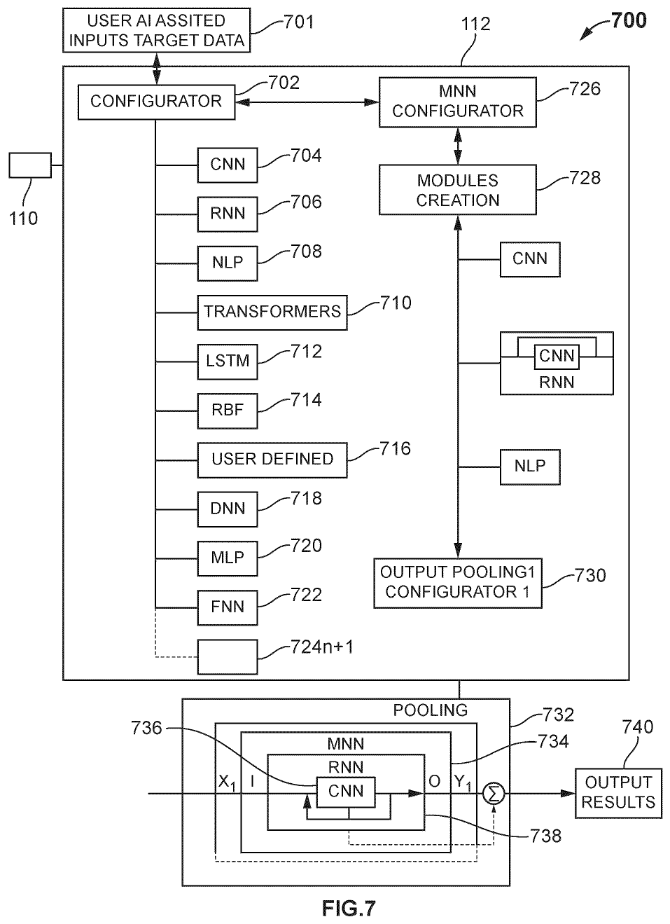

Referring now to FIG. 7, a block diagram illustrating an artificial intelligence system (AIS) 112 having a configurator 702 configured to select, execute, and embed a type of AI model to run based on either the data received or a user input, to form an assisted user input and via a trained data set and provide an output. An AI assisted neural net system using the AI module 112, user AI-Assisted target inputs are fed into the AI module 112. The AI configurator 702 is configured to examine the targeted inputs and based on the targets, it selects the appropriate AI or Neural Net model to deploy for each input. In operation, the module configurator creates neural network modules based upon each of the user AI assisted target data points and then embeds AI neural nets as required for accuracy. The outputs of the modules are then pooled based upon classification and the desired result and then returned to the user for visualization.

In operation, if the standardized data is a numerical data set, a neural net is deployed or embedded. For example, if the standardized data set is an image, a convolutional neural network 704 is deployed. If the standardized data set is a graph, a multilayer perceptron model 720 is deployed, and if the standardized data is a text, a natural language processing 708 with neural nets is deployed. Other models such as RNN 706, NLP 708, transformer 710, LSTM 712, RBF 714, user defined model 716, DNN 718, MLP 720, FN and 722 or AI 724n+1 may be executed by the configurator. As such, the configurator 702 is loaded with or in communication with a plurality of AI models and is configured to automatically choose the most optimized model based on a standardized data set type.

Once the data subset has been executed via the modules, the system is configured to send the output to pooling module 730 based upon classification and the desired result sent to output 740 for analysis and further processing as described with relation to FIG. 19.

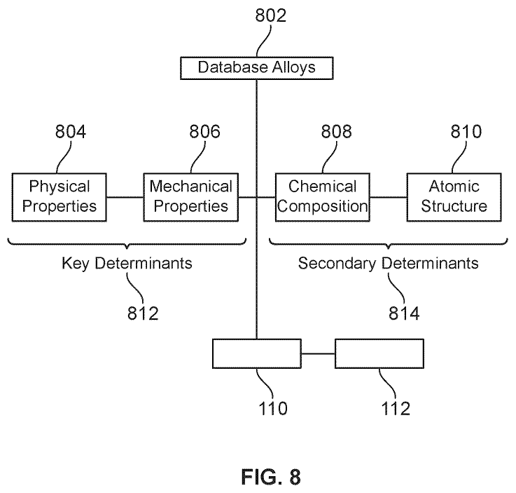

Referring now to FIG. 8, an exemplary second layer UI of the metal layer 800 is shown in greater detail. Metal database 802 is in communication with physical properties module 804, mechanical property module 806, chemical composition module 808 and atomic structure module 810. The physical properties module 804 may comprise a sub-data field of measurable properties such as electrical fields and the like, the mechanical properties 806 may comprise sub-data fields such as optimized particle size. These may be referred to as key determinates 812. This may comprise a sub-data field of measurable properties such as chemical resistance and the atomic structure module 810, which may comprise sub-data fields such as grain size, crystal structure and the like. These may be referred to as secondary determinates 814. Using these properties, the user may provide this second layer to a candidate compound search and provide for predictive addition of metals (e.g., sodium, potassium, magnesium, calcium, iron, zinc, copper, manganese, chromium, gold, silver, molybdenum and selenium and other metals) to the candidate compound based on similarities of other metals used in like-kind compounds. As an example, the system may be configured to target proteins and/or DNA to treat cancer, arthritis, parasitic and other diseases, with a special focus on the data collected, parsed, and organized by the system.

In an optional embodiment, the database 800 provides value to a “go/no go” decision making an actual elemental or engineering modification of the therapeutic, which can improve efficacy to reach the ideal therapeutic needed and is thus in communication with system 100 and visual display 216 for the user.

In one embodiment, the output of the software would be a modified receiver operator curve; the ordinate shall be a value generated from a regression combining available evidence for similar metals and would include a rating of how similar the innovator metal was concerning the existing metals. The abscissa may indicate the predictor of success, e.g., from 0 to 100. An additional characteristic may be considered to be the number of tests and data available for each comparison. For example, metals with only chemical resistance data would have a higher risk associated as a caution with the predictor compared, for example, with a chemical resistance, conductivity, density, and past uses cases.

In optional embodiments, a similar conductivity between extant and proposed use of the metal may indicate convergence and a probability of success, i.e., the closer the two values, the lower the risk and higher efficacy in its use as it relates to the desired clinical attribute. Likewise, as additional features and data are added, the greater the probability of success and the lower the risk and the higher the efficacy.

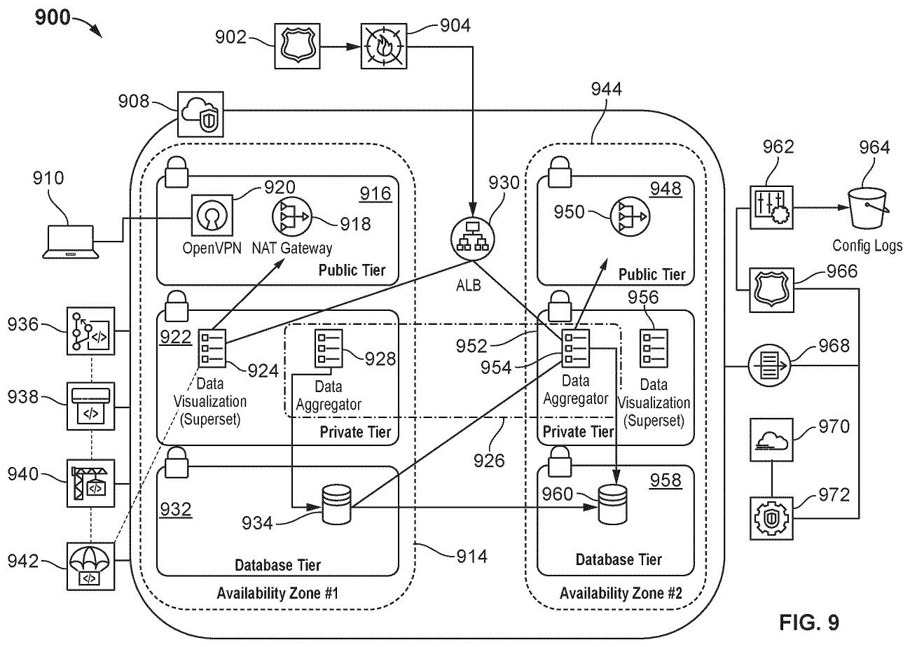

Referring now to FIG. 9, a block diagram of an exemplary system architecture and data routing scheme useable in an embodiment of the present invention, especially, a data aggregation service 900 defined in a virtual environment. Service 900 utilizes machine learning to scrape data from trusted web sources and export the aggregated data to a graph database or other database.

In operation, a user 910 or developer may input a request, in some embodiments, a request for data gathering, though this may also occur automatically. Subsequent the input, a scalable Domain Name System (DNS) 902, routes end users to certain applications and connects user requests to infrastructure running in the virtual networked environment such as, load balancers, or buckets (e.g., README file), and can also be used to route users to infrastructure outside of the virtual environment. The DNS is further configured to create a hosted zone to facilitate either creation of new DNS records or the migration of existing DNS records.

A web application firewall (WAF) 904 is in communication with the DNS 902 and is configured to monitors the HTTP(S) requests that are forwarded to a Cloud distribution or virtual network, an API Gateway REST API, an Application Load Balancer, or an AppSync GraphQL API. The WAF 904 enables control over access to content which is based on conditions specified, such as the IP addresses that requests originate from or the values of query strings, the service associated with a protected resource responds to requests either with the requested content or with an HTTP 403 status code (Forbidden). The data is then routed to a Virtual Private Cloud 906 that is configured to launch certain resources (or modules) into the virtual network.

The data is then routed to an application load balancer function 930 on the application layer, the seventh layer of the Open Systems Interconnection (OSI) model. After the load balancer 930 receives a request, it evaluates listener rules in priority order to determine which rule to apply, and then selects a target from the target group for the rule action. The AIS 112 is configured to provide its own listener rules to route requests to different target groups based on the standardized data and application traffic.

In operation, there are multiple availability zones 914 and 944 and the load balancer may route certain data to either availability zone. The availability zone 914 comprises a public tier 916, a private tier 922, and a database tier 932. The public tier 916 comprises a gateway 918 and an open VPN 920. The gateway 918 is in communication with code deploy 942 and the open VPN 920 is in communication with the user machine 910. The public tier 916 is configured as a public subnet that can send outbound traffic directly to the internet.

The private tier 922 comprises data aggregator 928 and data visualization module 924. The data aggregator 928 is configured to execute machine learning functions, via AIS 112, configured to scrap and collect data from trusted web sources. Data visualization 924 allows the user to explore and visualize data. It is also configured to allow the user to choose web server (e.g., Gunicorn®, Nginx®, Apache®), metadata database engine (MySQL, Postgres, MariaDB, etc.), message queue (Redis, RabbitMQ, SQS, etc.), results, backend (S3, Redis, Memcached, etc.), caching layer (Memcached, Redis, etc.)

The data visualization module 924 and data aggregator 928 are in communication with the database tier 932. The database tier comprises database 934 configured as fully managed database service built for the cloud that can build and run graph applications and is optimized for storing billions of relationships and querying the graph with milliseconds latency.

Availability zone 944 is in communication with the load balancer and comprises public tier 948, private tier 956, and database tier 960. The public tier 948 comprises gateway 950 which is configured to act in the same way as gateway 918 private tier 952 comprises data aggregator 954 and data visualization module 956 each of which are also configured to act in the same way as data visualization module 924 in data aggravator 928 and lastly, the database tier 958 comprises database 960 that operates in a similar fashion to database 934.

The virtual network 908 is in communication with source 910 to be created with the appropriate source material to build and deployment stage for data gathering. The source 936 is configured to communicate with the code repositories comprising GitHub 936, Bitbucket 938, CodeCommit 940, or Gitlab 942. Code Pipeline is configured to create a CloudFormation change set after Code Build finishes the build stage and later executes that change set with or without manual approval, Code Build is configured with an appropriate buildspec.yml file to build and package the code that needs to be deployed. The build step is triggered by Code Pipeline after source changes and before deployment begins. In the case of serverless, Code Build is configured to build the code (if needed), create a CloudFormation template package, and pass it along to Code Pipeline to create a CloudFormation change set and then execute on the change set. Code Deploy 942 is configured to automate code deployments to the cloud. Deployment groups are created for each code base and configured to deploy specific code updates to their corresponding computer architectures.

Config logs 964 are configured as KMS Encrypted buckets to store logs from Config 962. The bucket is configured with a dedicated bucket policy only allowing access to Config 962 to read/write logs. Lifecycle Policy is configured to delete logs after some period of time, usually 30 days. DNS Queries 966 is configured as protective firewall, and CloudTrail is deployed in all regions to log, continuously monitor, and retain account(s) activity related to API actions across the infrastructure. Threat detection 972 continuously monitors malicious activity and unauthorized behavior to protect account(s), workloads, and data.

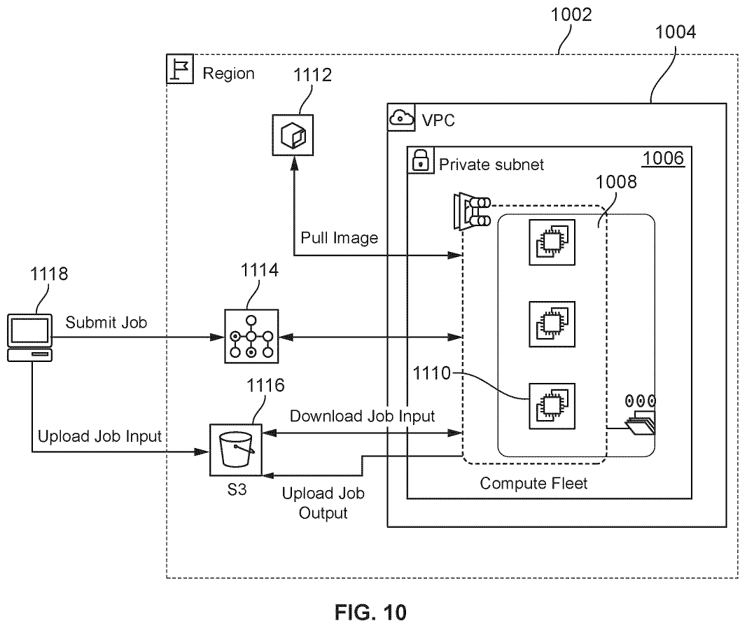

FIG. 10 is block diagram of an exemplary virtual network for use in embodiments of the present invention. In operation, the network is set up in regions 1002 that are designed to be isolated from each other region. The virtual cloud 1004 is generated and enables you to resource into a virtual network that the user or the system defines. Private Subnet 1006 ensures the user cannot route outbound traffic directly to the internet.

Fleet 1008 is deployed and is configured to launch multiple instance types across multiple with a single API. ECR 1112 comprises repositories that are created to house container images in the primary operating region. Batch 114 is a fully managed batch computing service that plans, schedules, and runs your containerized batch or ML workloads across the full range of the virtual network. Cloud object storage 1116 is in communication with the computer fleet for data transmission after job 1118 is submitted.

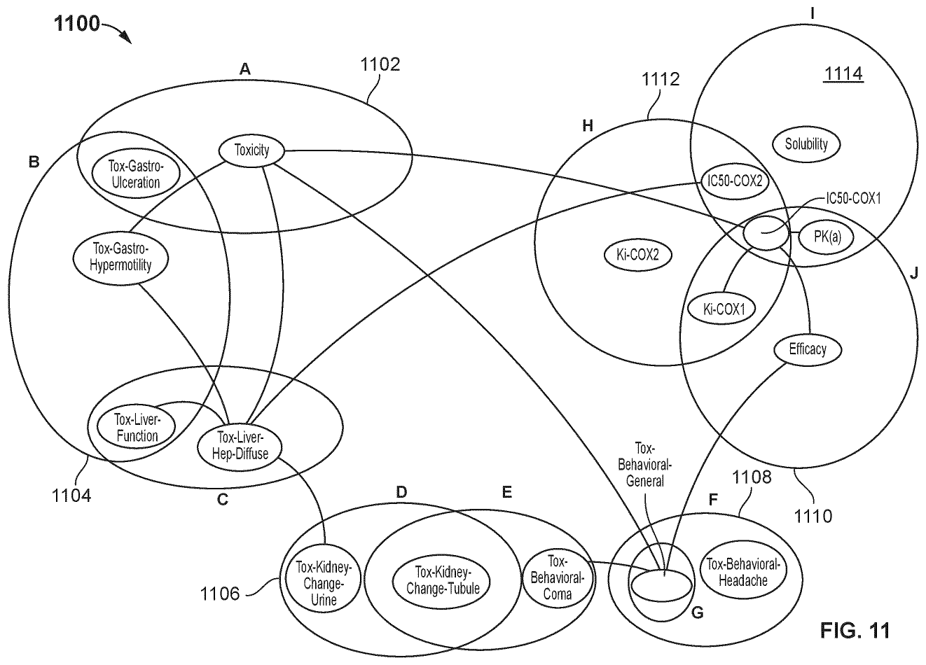

FIG. 11 is an exemplary graph 1100 produced by the AIS 112 and data visualization module 924 and data aggregator 928 results for [please describe results and what this graph tells us]. Paul, please help. FIG. 11 is an exemplary graph 1100 an output of a graph database software that depicts the relationship between a molecules characteristic for extremely fast queries.

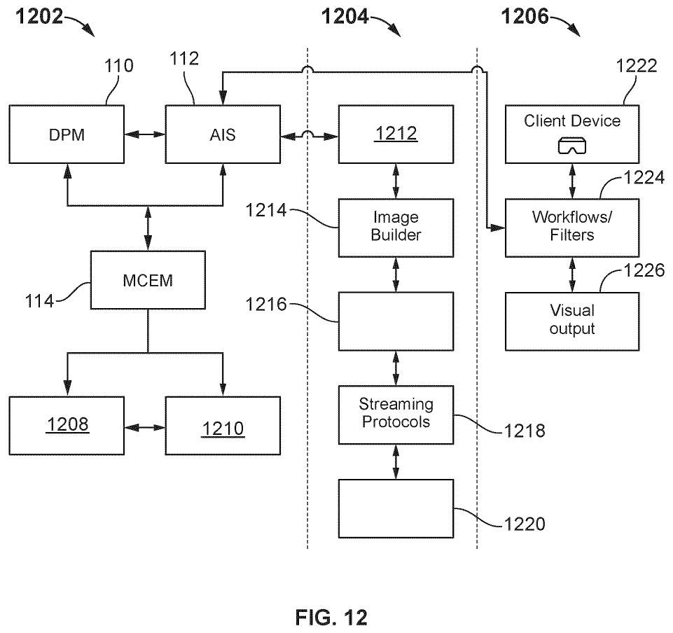

FIG. 12 is a block diagram illustrating modes of data gathering, transformation and building an interactive environment in one embodiment of the present invention. In embodiments, the system is tri-furcated comprises tools and modules to gather, sort and transform data phase 1202, comprises tools and modules to use the transformed data and turn them into data usable by a ARVR machine phase 1204, and tools and modules to allow the user to use the interactive environment and graphical representations and change molecules and compounds in an interactive way for a practical application in one example, drug discovery, phase 1206. In the first phase 1202, the DPM 110 AIS 112 and MCEM 114 are in communication with batch 1208 and AI training module 1210. AI training module 1210 is configured to store compiled standardized data, in this case, training data, and use this compiled dataset to train and improve the data aggregation model. In this way, the module 1210 creates a processing job for the standardized data, then creates a training job to train the module, and finally, the trained module is evaluated against the success criteria and registered in the module registry as a pickle file where it can be accessed and approved by data scientists, initially. Once approved, the machine learning will automatically process the data to train data sets using a recurrent neural network model to form a link for convergence. The AIS 112 is configured to automatically choose the model based on the type of standardized data. If the standardized data, in embodiments, is a numerical data set, a neural net may be deployed or embedded, if the standardized data set is an image, a convolutional neural network may be deployed or embedded, if the standardized data set is a graph, a multi-layer perception model may be deployed or embedded and if the standardized data set is text, a natural language processing with neural net may be deployed or embedded. It should be noted that the AIS 112 is in further communication with phase 1204 in phase 1206 to deploy machine learning models to certain aspects and modules contained therein.

Phase 1204 receives data sets from the MCEM 114 and AIS 112 at a rendering module 1212. The rendering module 1212 together with image builder 1214 engine 1216 and streaming protocol 1218 and database 1220 an interactive AR/VR environment is generated and comprises graphical representations of the candidate molecules in atomic resolution based on a user query. The rendering module is configured with 3-D rendering software and may be configured to use an image builder 214 that provides 2-D and 3-D platform to create scenes. The engine 216 is configured as a real time 3-D creation for photo-real visuals and immersive experiences and is configured to build artifacts that will be fully managed on a non-persistent application screaming service 1218.

This data can then be accessed at phase 1206 and viewed via client device 1222 using workflow filters 1224 and visual output 1226. Based on a received signal, that may be AI assisted, the user may alter a configuration of the candidate compound and interact with a candidate compound and replace a molecule in the Kennedy compound. The system is then configured to automatically provide clinical attribute data and predictive scores as to whether that molecule will meet the needs of the user for a specific drug, for example. A user interface generates clinical characteristics of the candidate compound as altered by the user in near real time.

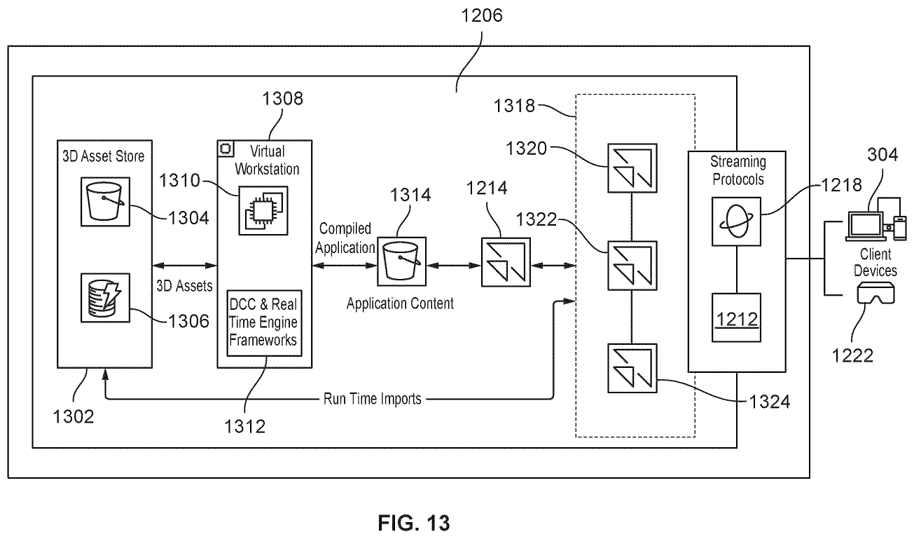

FIG. 13 is a block diagram of an exemplary system architecture for building a virtual interactive environment in embodiments of the present invention is provided. On the virtual network 1206 there may be a 3DS store 1302 comprising an asset bucket and an asset table 1304 and 1306 respectively. The 3-D assets may be sent to a virtual workstation 1308 comprising graphic instances 1310 and real time engine framework 1312. Compiled assets and graphics are then sent to bucket 1314 at which point it can be retrieved by image builder 1214. The images are then sent to a fleet 1318 consisting of streaming instances 1324 that run the images that are specified in a stack 1320 and 1322 which consists of associated fleet user policies and storage configurations. Streaming protocols 1215 are configured to output the images to the client device.

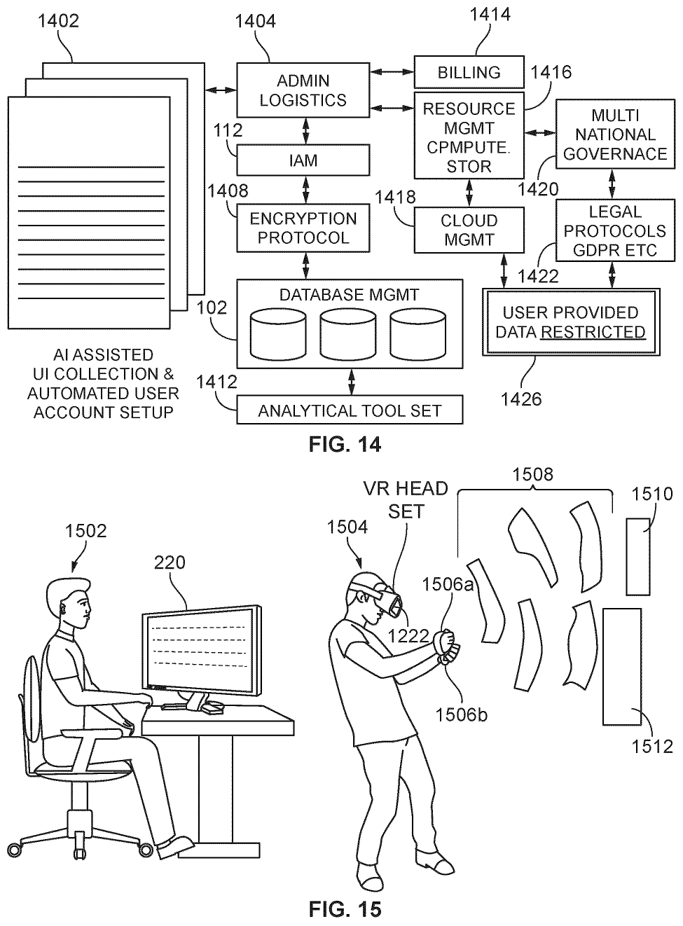

FIG. 14 is a block diagram of an exemplary user management architecture in embodiments of the present invention. In this embodiment, servers 1402 are in communication with admin logistics 1404, AIM 112, encryption protocol 1408, databases 102 analytical tool set 1214 building 1414, resource management computer store 1416, cloud management 1418, multinational governments 1420, legal protocols 1422, and use restrictions 1426. In operation, these services allow organizations to communicate with the virtual network and ensure proper data protection, encryption, and management.

Now with reference to FIG. 15, an exemplary illustration of a user interacting with a user interface on a computer and a user interacting with the interactive environment in embodiments of the present invention is provided. In one embodiment, there may be a user 1502 at a computer user interface 220. In this way, normalized desktop features may be used for predictive candidate compounds as recited in relation to FIGS. 1 through 14. In embodiments, a VR user 1504 may use a VR headset 1222 to interact in a VR/AR/mixed reality environment using hand trackers 1506A in 1506B. Images 1508 1510 and 1512 can be manipulated based on signals received from the hand toggles 1506A in 1506B

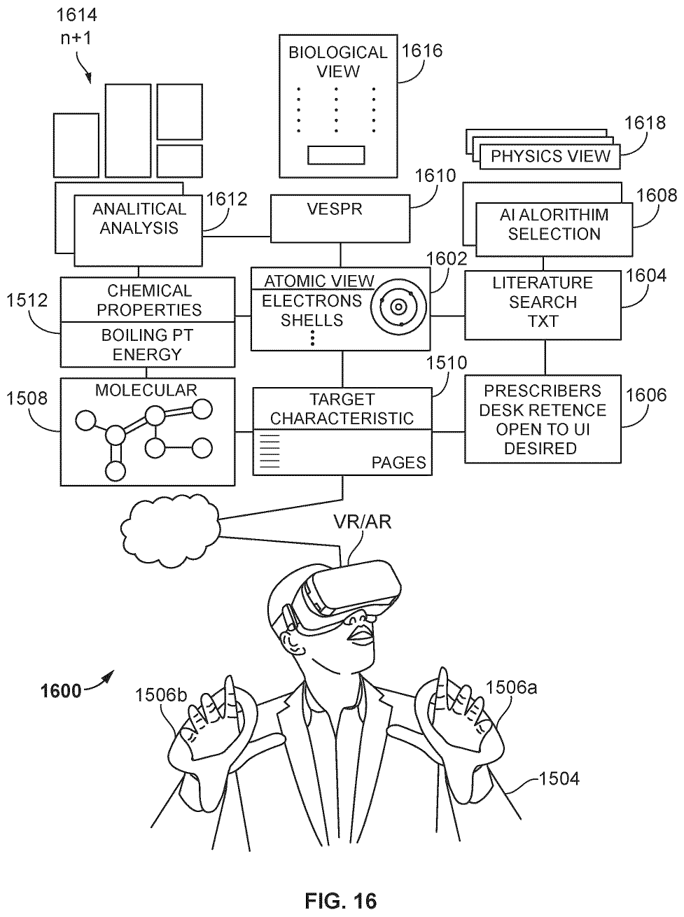

FIG. 16 is another exemplary illustration of a user interacting with the interactive environment in embodiments of the present invention. This figure shows examples of different drop-down boxes that can be viewed with the VR/AR components. In operation, the hand trackers 1506A and 1506B allow the user to toggle through a plurality of different modes 1614n+1. Example properties that the user can choose are shown, for example, analytical analysis 1612, chemical properties 1512, molecular bonding angles 1508, target characteristics 1510, atomic view 1602, vesper 1610, biological view 1616, and physics view 1618. In operation, if a user chooses any of these particular modes, a new user interface will be generated and populate the VR/AR screen and allow the user to interact with that environment.

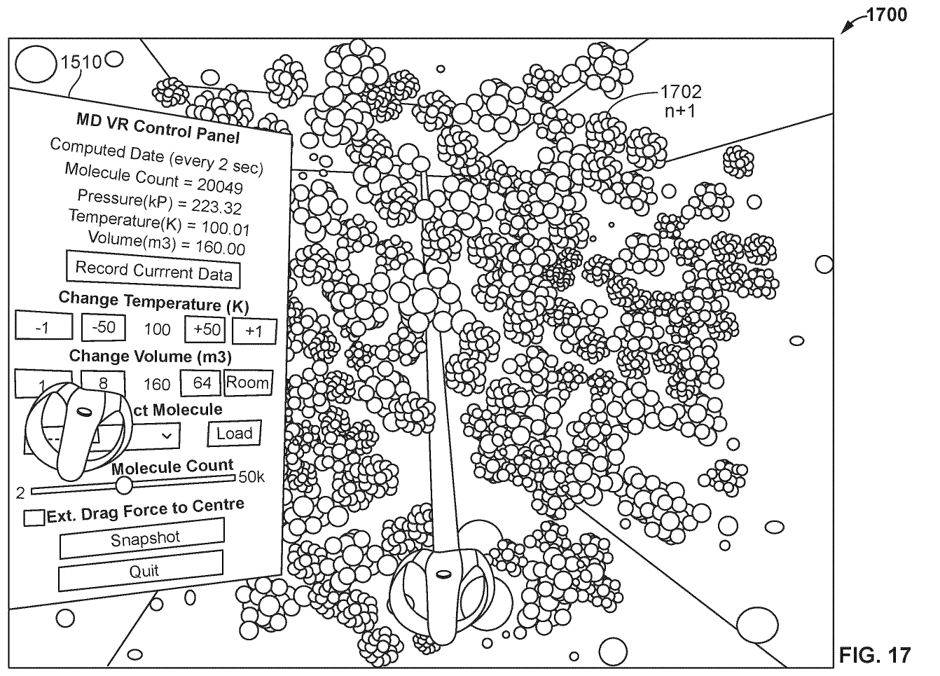

FIG. 17 is an exemplary user interface showing an interactive molecule and user interface with selectable parameters in embodiments of the present invention. As shown, control panel 1510 is provided with different molecule or substance parameters that can be chosen by a user using the hand trackers 1506a and 1506b. Representations of molecules and compounds are shown in 1700 and comprise molecules 1702n+1. The molecules are similar to classic ball and stick model but in a simulated form such that the user can interact with them in an AR/VR environment.

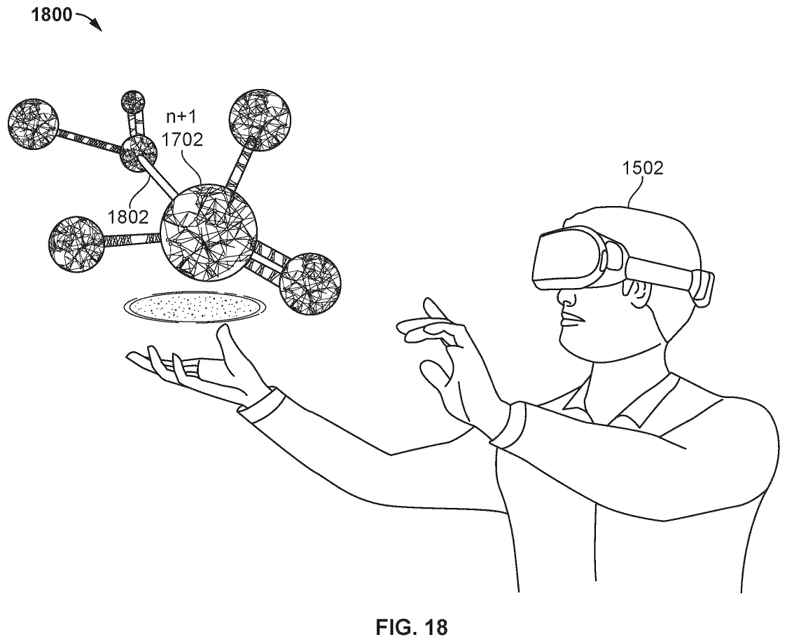

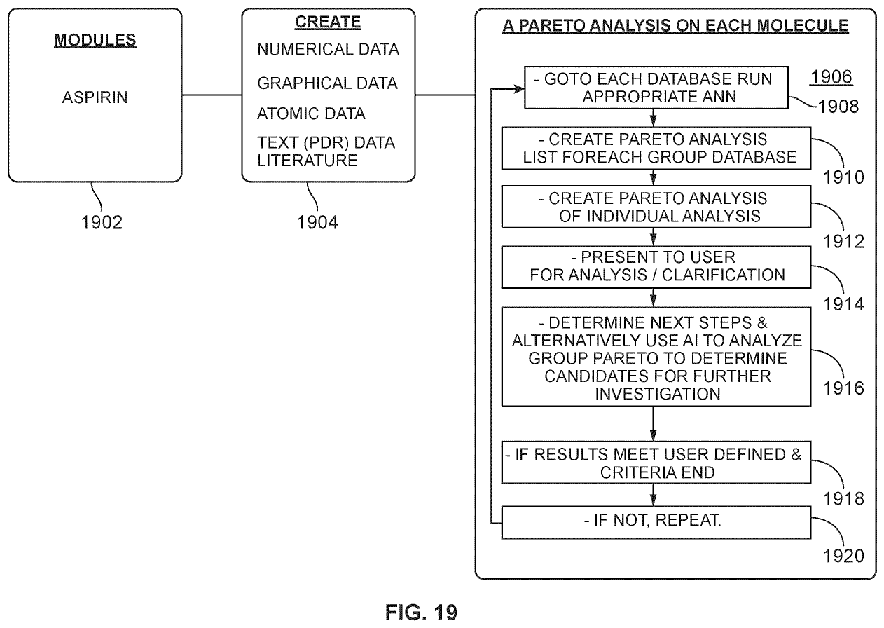

FIG. 18 is an illustration of a user interacting in the environment with a molecule and compound in embodiments of the present invention. In operation, the user can see bond angles and different spaces on an atomic and subatomic level to make a decision whether to try a different molecule based on predictive analyses and suggestions by the system or based on what the user can see with the molecule and bonding 1802.

FIG. 19 is a flow chart showing a recommendation system in embodiments of the present invention. In this embodiment, a means module 1902 contains the target molecule(s) and or characteristic(s) that the user has selected for investigation and is in communication with a create module 1904. The create module examines the request and determines the appropriate databases to be used in the discovery process. The analysis module 1906 is in communication with the create module 1904. The analysis module first step 1908 determines the appropriate Artificial Intelligence technique based upon the data type/classification/category for each data set for each molecule and creates a process flow and input for the results visualization(s) based upon the users' inputs. The output of 1908 communicates with 1910 for creating a prioritize list for each AI module based on the users desired focus. The outputs of 1910 are then fed to 1912 wherein the pareto analysis outputs are collected and formatted for 1914 where the user will be able to see the results in whatever format requested. Options to change the format can also be introduced at this time and or for additional format viewing methods. At this juncture, 1914, communicates with 1916 that provides AI suggestions for next steps and provides options for the user. A decision tree, 1918 provides the users choices and if desired results have been met, the process ends. If the conditions for decisions have not been met, 1918 communicates with 1920 and the process repeats.

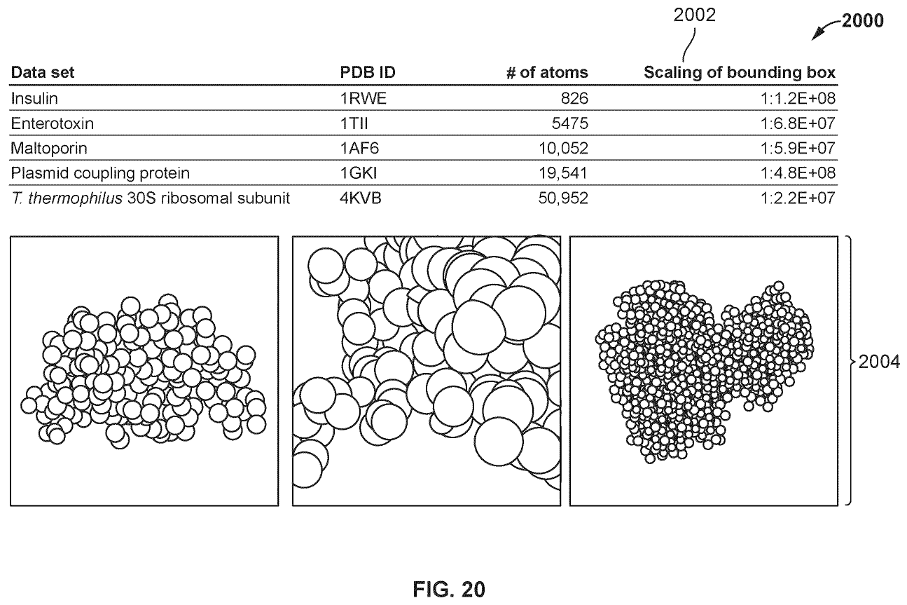

Referring now to FIG. 20, an exemplary user interface showing an interactive molecule and user interface with a data set provide by the system in embodiments of the present invention is shown in 2000. In this embodiment, the system provides a plurality of data shown in 2002 based on a candidate molecule or compound selected such as number of atoms and scaling of bounding box. Visuals 2004 may also be provided in this exemplary interface.

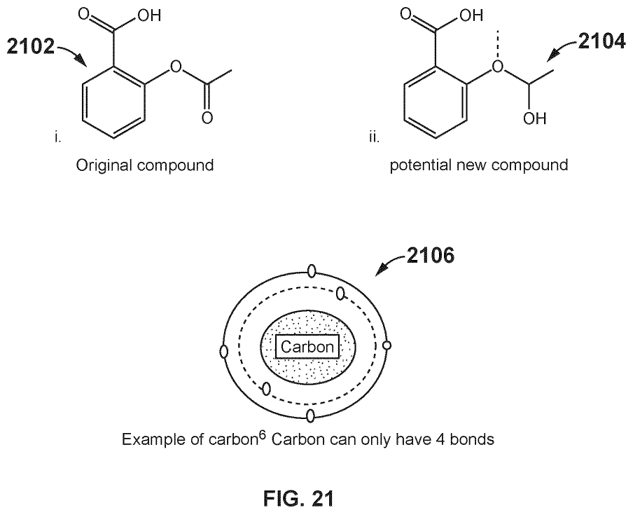

Referring now to FIG. 21, a molecular diagram showing a compound and candidate compound together with a bonding map in embodiments of the present invention is shown. In this embodiment, using the system, the user can ascertain if the candidate molecule has been properly aligned with respect to other properties of the compound. When running an in-silico assay for effectiveness, the system will notify or show the user if the compound has been adjusted properly/correctly. In the compound shown in 2102, the system is able to identify, for the user, the total count of protons, total count and electrons, and total count in neutrons and then for the potential new compound, identify the total count of protons, total count of electrons, and total count of neutron as well also showing the bonding potential 2106 for the compound in total. As an example, the charge of an element is the number of protons minus the number of electrons, and thus, the system is configured to fully track each sub-part, compare symmetrical/asymmetrical bonds for the two or more compounds, and run the simulation (VR/AR).

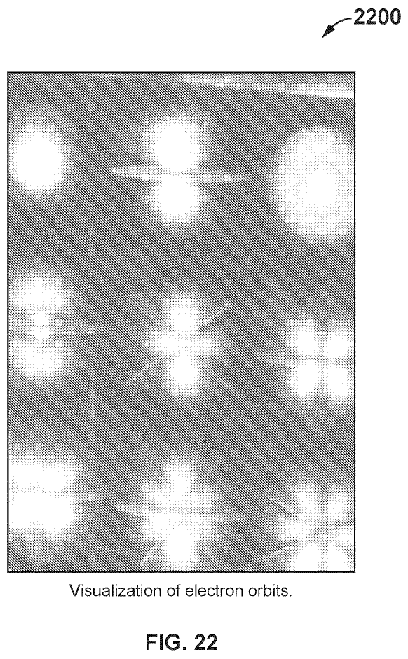

Referring now to FIG. 22, an electron orbit map showing subatomic visualization in a user interface and embodiment of a present invention is shown at 2200. As shown, the system allows the visualization of optimized adherence points using VR/AR to track user-specific characteristics in a three-dimensional environment and to evaluate optimization, bonds (both positive and negative effects), and determine if, by slight rotation or modification of position, a stronger, more stable, more effective relationship can be achieved.

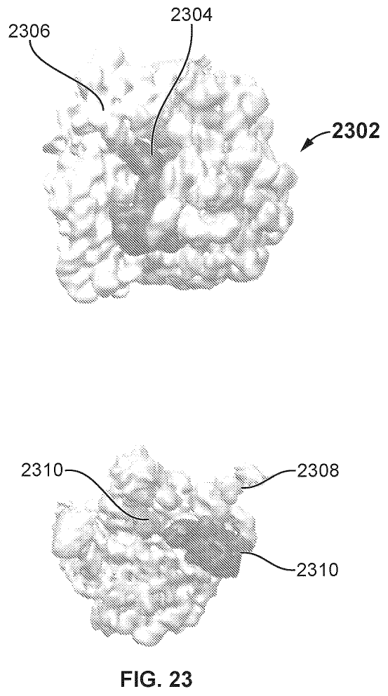

Referring now to FIG. 23 an exemplary compound visualization showing potential complexes and exemplary UIs in embodiment of the present invention. In this example, 70S·Tet(O)·GDPNP·fMet-tRNA complex 2302 is shown with various density for the 50S subunit with Tet(O) and P-site tRNA.) The user can view, interact, and change certain aspects using VR/AR and the system will provide predictive results.

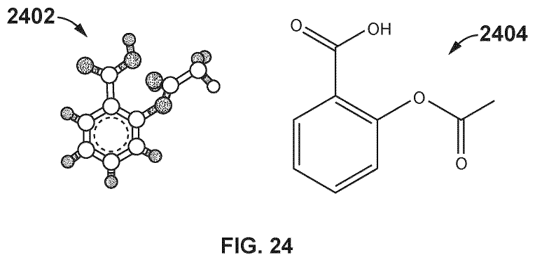

FIG. 24 is an exemplary molecular diagram showing how an insertion of a candidate molecule may cause a condition that is realized by the system in embodiments of the present invention at 2402 and 2404. The system is configured to provide predictive results as to certain factors such as blood clotting and lung disease, then test all blood types as the user requires.

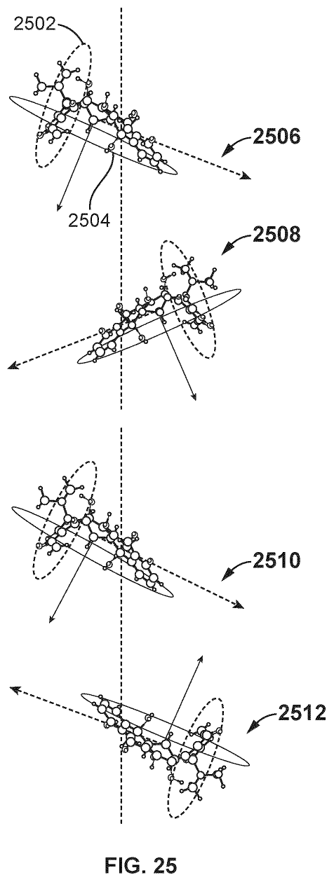

FIG. 25 is a molecular diagram showing symmetrical and asymmetrical chemical bonding in embodiments of the present invention. As shown, each of the angles 2506 2504 25082510 and 2512 show a variety of different bond angles the user can visualize and make decisions based on the angles and what other molecules may fit.

In exemplary embodiments, this approach to scoring is objective and appreciably better than human judgment in terms of accuracy and efficiency. It removes any subjective input in favor of more objective appraisals when applied to the selection process and go/no go decisions in metal development for all industry sizes.

Advantageously, the present invention makes it possible to make decisions early, saving time and expenses. Further, the data supports management decisions with more circumspection. The system compares the known physical, chemical, genetic, and biological dimensions of molecules and metals and identifies similarities in their composition and behavior. The system performs these comparisons on any level of scale, in vivo, in vitro, and silica simultaneously. In addition, the system self-checks for the accuracy of results using available data repositories. Further, the system uses machine learning to train the algorithms to accurately identify the high potential similarity matches and display results using 3D, virtual, or augmented reality visualizations.

Preferred embodiments of this invention are described herein, including the best mode known to the inventors for carrying out the invention. It should be understood that the illustrated embodiments are exemplary only and should not be taken as limiting the scope of the invention.

The preceding description comprises illustrative embodiments of the present invention. Having thus described exemplary embodiments of the present invention, it should be noted by those skilled in the art that the within disclosures are exemplary only and that various other alternatives, adaptations, and modifications may be made within the scope of the present invention. Merely listing or numbering the steps of a method in an order does not constitute any limitation on the order of the steps of that method. Many modifications and other embodiments of the invention will come to mind to one skilled in the art to which this invention pertains, benefiting the teachings in the preceding descriptions. Although specific terms may be employed herein, they are used only in a generic and descriptive sense and not for purposes of limitation. Accordingly, the present invention is not limited to the specific embodiments illustrated herein.

What is claimed is:

1. A computing system for evaluating candidate molecules for use in candidate compound discovery, the computer system comprising:
a non-transitory computer-readable memory; and
a processor configured to execute instructions stored on the non-transitory computer-readable memory which, when executed, cause the processor to:
receive a type of standardized data from at least a database, wherein the standardized data is for compounds, metals, molecules, atoms, sub-atomic particles or any combination thereof, and wherein the standardized data types comprise numerical data sets, images, graphs, text, or any combination thereof;
receive a user query, wherein the user query comprises a request for a desired attribute for the candidate compound;
process each of the standardized data types with one or more trained machine learning models, wherein the one or more machine learning models are configured to recommend at least one of a plurality of the candidate molecules to be bonded with the candidate compound based on the user query;
generate an interactive environment comprising graphical representation of the candidate molecule and candidate compound based on the user query;
based on a received a signal from a user, alter a configuration of the candidate compound to allow the user, using virtual reality (VR), augmented reality (AR) or both, to interact with the candidate compound and the plurality of candidate molecules, wherein based on a received additional signal from the user, further alter the candidate compound with another of the plurality of molecules; and
generate clinical characteristics of the candidate compound based on the further alteration by the user.

2. The system of claim 1, wherein the instructions, when executed, further cause the processor to:
based on the type of the standardized data received, select one of the one or more machine learning models to output a trained data set, wherein:
if the standardized data is the numerical data set, execute a neural net model;
if the standardized data set is an image, execute a convolutional neural network model;
if the standardized data set is a graph, execute a multilayer perceptron model; and
If the standardized data is a text, execute a natural language processing with neural nets model.

3. The system of claim 2, wherein the instructions, when executed, further cause the processor to:
pool the trained data sets and combine the sets; and
perform a Pareto analysis to output a predicted most accurate of the data sets.

4. The system of claim 3, wherein the instructions, when executed, further cause the processor to:
further process the trained data sets using a recurrent neural network model to form a loop for convergence.

5. The system of claim 1, wherein the standardized data comprises bonding angles, electron, proton and neutron configurations, melting point, toxicity, physical characteristic, chemical characteristic, atomical characteristic, biological dimensions, or any combination thereof.

6. The system of claim 1, wherein the instructions, when executed, further cause the processor to load normalize the standardized data received.

7. The system of claim 1, wherein the instructions, when executed, further cause the processor to:
transform the standardized data into numerical data sets; and
tag, index and assign a value to each of the data set based on the user query.

8. The system of claim 3, wherein the instructions, when executed, further cause the processor to:
generate, using a neural net, a first user interface;
subsequent the user input, load a menu on the user interface with recommended molecules based on the user input using the neural net; and
order the recommended molecules in the menu based on a predictive success parameter using the trained data sets.

9. The system of claim 8, wherein the instructions, when executed, further cause the processor to:
load a second layer menu on the first user interface;
order the recommended metals on the second layer menu based the predictive success parameter using the trained data sets.

10. The system of claim 9, wherein the instructions, when executed, further cause the processor to:
define a virtual network configured to facilitate communication between at least a database and a plurality of servers having the processor and the memory in communication therewith.

11. The system of claim 1, wherein the instructions, when executed, further cause the processor to:
When generating the interactive environment, further generate a caching layer to construct a spider graph, radar chart, or both.

12. The system of claim 1, wherein the instructions, when executed, further cause the processor to:
receive a request at a load balancer, and evaluate listener rules in a priority order to determine which of the listener rules to apply;
select a target from a target group for listener rule to route requests to different target groups based on the content of application traffic;
execute a cloud deep learning model to run a three-dimensional rendering script to produce the interactive environment comprising the candidate molecule in atomic resolution.

13. The system of claim 12, wherein the instructions, when executed, further cause the processor to:
create on-demand instance and a plurality of stacks for a streaming application and associate a fleet comprising a plurality of streaming instances, wherein the stack comprises an associated fleet to produce the candidate molecule and corresponding sub-atomic particles in a sub-atomic visualization.

14. A computer-implemented method candidate compound discovery, comprising executing on a processor the steps of:
receiving a type of standardized data from at least a database, wherein the standardized data is for compounds, metals, molecules, atoms, sub-atomic particles or any combination thereof, and wherein the standardized data types comprise numerical data sets, images, graphs, text, or any combination thereof;
receiving a user query, wherein the user query comprises a request for a desired attribute for the candidate compound;
processing each of the standardized data types with one or more trained machine learning models, wherein the one or more machine learning models are configured to recommend at least one of a plurality of the candidate molecules to be bonded with the candidate compound based on the user query;
generating an interactive environment comprising graphical representation of the candidate molecule and candidate compound based on the user query;
based on a received a signal from a user, altering a configuration of the candidate compound to allow the user, using virtual reality (VR), augmented reality (AR) or both, to interact with the candidate compound and the plurality of candidate molecules, wherein based on a received additional signal from the user, further alter the candidate compound with another of the plurality of molecules; and
generating clinical characteristics of the candidate compound based on the further alteration by the user.

15. The method of claim 14, further comprising:
based on the type of the standardized data received, selecting one of the one or more machine learning models to output a trained data set, wherein:
if the standardized data is the numerical data set, execute a neural net model;
if the standardized data set is an image, execute a convolutional neural network model;
if the standardized data set is a graph, execute a multilayer perceptron model; and
If the standardized data is a text, execute a natural language processing with neural nets model.

16. The method of claim 15, further comprising:
pooling the trained data sets and combine the sets; and
performing a Pareto analysis to output a predicted most accurate of the data sets.

17. The method of claim 14, further comprising:
generating, using a neural net, a first user interface;
subsequent the user input, loading a menu on the user interface with recommended molecules based on the user input using the neural net; and
ordering the recommended molecules in the menu based a predictive success parameter using the trained data sets.

18. The method of claim 17 further comprising defining a virtual network configured to facilitate communication between at least one database and a plurality of servers having the processor and the memory in communication therewith.

19. The method of claim 14, further comprising:
receiving a request at a load balancer, and evaluate listener rules in a priority order to determine which of the listener rules to apply;
selecting a target from a target group for listener rule to route requests to different target groups based on the content of application traffic;
executing a cloud deep learning model to run a three-dimensional rendering script to produce the interactive environment comprising the candidate molecule in atomic resolution.

20. A non-transitory computer-readable medium having stored thereon program instructions that, upon execution by a computing device, cause the computing device to perform operations comprising:
receive a type of standardized data from at least a database, wherein the standardized data is for compounds, metals, molecules, atoms, sub-atomic particles or any combination thereof, and wherein the standardized data types comprise numerical data sets, images, graphs, text, or any combination thereof;
receive a user query, wherein the user query comprises a request for a desired attribute for the candidate compound;
process each of the standardized data types with one or more trained machine learning models, wherein the one or more machine learning models are configured to recommend at least one of a plurality of the candidate molecules to be bonded with the candidate compound based on the user query;
generate an interactive environment comprising graphical representation of the candidate molecule and candidate compound based on the user query;
based on a received a signal from a user, alter a configuration of the candidate compound to allow the user, using virtual reality (VR), augmented reality (AR) or both, to interact with the candidate compound and the plurality of candidate molecules, wherein based on a received additional signal from the user, further alter the candidate compound with another of the plurality of molecules; and
generate clinical characteristics of the candidate compound based on the further alteration by the user.
